(19) United States
(12) Patent Application Publication
(10) Pub. No.: US 2003/0194701 A1
(43) Pub. Date: Oct. 16, 2003

(54) Diffuse large cell lymphoma diagnosis and outcome prediction by expression analysis

(76) Inventors: Todd R. Golub, Newton, MA (US); Pablo Tamayo, Cambridge, MA (US); Margaret Shipp, Wellesley, MA (US); Eric S. Lander, Cambridge, MA (US); Jon C. Aster, Lexington, MA (US)

Correspondence Address: Lisa M. Treannie, Esq., HAMILTON, BROOK, SMITH & REYNOLDS, P.C., 530 Virginia Road, P.O. Box 9133, Concord, MA, 01742-9133, US

(21) Appl. No.: 09/989,758

(22) Filed: Nov. 20, 2001

Related U.S. Application Data
(60) Provisional application No. 60/252,142, filed on Nov. 20, 2000.
(60) Provisional application No. 60/254,458, filed on Dec. 8, 2000.

(51) Int. Cl. C12Q 1/68; G01N 33/53

(52) U.S. Cl. 435/6; 435/7.1

(57) ABSTRACT

Methods for predicting phenotypic classes of lymphomas, such as lymphoma type or treatment outcome, for lymphoma samples based on gene expression profiles are described.

### RELATED APPLICATIONS

This application claims the benefit of U.S. Provisional Application No. 60/252,142, filed on Nov. 20, 2000, and U.S. Provisional Application No. 60/254,458, filed on Dec. 8, 2000. The entire teachings of the above applications are incorporated herein by reference.

### GOVERNMENT SUPPORT

The invention was supported, in whole or in part, by grant 1PO1 CA6696-01A1 from the National Institutes of Health. The Government has certain rights in the invention.

### BACKGROUND OF THE INVENTION

Classification of biological samples from individuals is not an exact science. In many instances, accurate diagnoses and safe and effective treatment of a disorder depend on being able to discern biological distinctions among morphologically similar samples, such as, for example, tumor samples. The classification of a sample from an individual into particular disease classes has proven to be difficult, incorrect or equivocal. Typically, using traditional methods such as histochemical analyses, immunophenotyping and cytogenetic analyses, only one or two characteristics of the sample are analyzed to determine the sample's classification, resulting in inconsistent and sometimes inaccurate results. Such results can lead to incorrect diagnoses and potentially ineffective or harmful treatment. Furthermore, important biological distinctions are likely to exist that have yet to be identified due to the lack of systematic and unbiased approaches for identifying or recognizing such classes. Thus, a need exists for an accurate and efficient method for identifying biological classes and classifying samples.

### SUMMARY OF THE INVENTION

The present invention relates to one or more sets of informative genes whose expression levels correlate with a class distinction between samples. In a particular embodiment, the class distinction is a lymphoma class distinction, such as a Non-Hodgkin's lymphoma class distinction (e.g., folicular lymphoma (FL) or diffuse large cell lymphoma (DLCL)). In another embodiment the class distinction can be a treatment outcome or survival class distinction.

When classifying a sample as to its source, for example FL or DLCL, informative genes can be, for example, all or a subset of the genes shown in FIGS. 3A and 3B and/or the genes shown in FIGS. 4A and 4B. FIGS. 3A and 3B show informative genes whose expression is increased in DLCL and decreased in FL. FIGS. 4A and 4B show informative genes whose expression is decreased in DLCL and increased in FL.

When classifying a sample into a DLCL treatment outcome class, for example, informative genes can be, for example, all or a subset of the genes shown in FIG. 1 and/or the genes shown in FIGS. 2A and 2B. FIG. 1 shows informative genes whose expression is increased in low risk (i.e., positive treatment outcome) and decreased in high risk individuals. FIGS. 2A and 2B show informative genes whose expression is decreased in low risk and increased in high risk individuals.

The invention relates to a method of classifying a sample according to lymphoma type comprising the steps of isolating a gene expression product from at least one informative gene from one or more cells in said sample; and determining a gene expression profile of at least one informative gene, wherein the gene expression profile is correlated with a lymphoma type, thereby classifying the sample with respect to lymphoma type. In one embodiment, the lymphoma type is diffuse large cell lymphoma. In another embodiment, the lymphoma type is follicular lymphoma.

In one embodiment of the method, the gene expression product is mRNA, and in a particular embodiment, the gene expression profile is determined utilizing hybridization probes specific to one or more informative genes. In particular, the gene expression profile is determined utilizing oligonucleotide microarrays, containing probes or primers for all or a subset of the informative genes disclosed herein, immobilized on a solid support chip. In another embodiment of the invention, the gene expression product is a peptide, and in a particular embodiment, the gene expression profile is determined using antibodies.

The invention also relates to a method of assigning a sample to a lymphoma class, comprising the steps of determining a weighted vote for one of the classes of one or more informative genes in said sample in accordance with a model built with a weighted voting scheme, wherein the magnitude of each vote depends on the expression level of the gene in said sample and on the degree of correlation of the gene's expression with class distinction; and summing the votes to determine the winning class, wherein the winning class is the lymphoma class to which the lymphoma sample is assigned. In one embodiment, the weighted voting scheme is V_{g}=a_{g} (x_{g}−b_{g}), wherein V_{g} is the weighted vote of the gene, g; a_{g} is the correlation between gene expression values and class distinction; b_{g}=(μ₁(g)+μ₂(g))/2 which is the average of the mean log₁₀ expression value in a first class and a second class; x_{g} is the log₁₀ gene expression value in the sample to be tested; and wherein a positive V value indicates a vote for the first class, and a negative V value indicates a vote for the second class.

The invention further relates to a method of classifying a sample according to predicted treatment outcome comprising the steps of isolating a gene expression product from at least one informative gene from one or more cells in said sample; and determining a gene expression profile of at least one informative gene, wherein the gene expression profile is correlated with a treatment outcome, thereby classifying the sample with respect to treatment outcome. In one embodiment the sample is a Non-Hodgkin's lymphoma sample, e.g., a DLCL sample. In one embodiment, the gene expression product is mRNA. In one embodiment the gene expression profile is determined using hybridization probes specific to one or more informative genes, and in a particular embodiment the gene expression profile is determined using oligonucleotide microarrays. In another embodiment the gene expression product is a peptide, and in a particular embodiment the gene expression profile is determined using antibodies. In one embodiment the predicted treatment outcome is survival after treatment. In another embodiment the informative gene is a gene shown in FIGS. 1, 2A and 2B.

The invention further relates to a method of assigning a Diffuse Large Cell Lymphoma (DLCL) sample to a treatment outcome class, including the steps of determining a weighted vote for one of the classes of one or more informative genes in the sample in accordance with a model built with a weighted voting scheme, such that the magnitude of each vote depends on the expression level of the gene in the sample and on the degree of correlation of the gene's expression with class distinction; and summing the votes to determine the winning class, such that the winning class is the treatment outcome class to which the DLCL sample is assigned.

In one embodiment the weighted voting scheme is:

V_{g}=a_{g}(x_{g}−b_{g}),

wherein V_{g} is the weighted vote of the gene, g; a_{g} is the correlation between gene expression values and class distinction; b_{g}=(μ₁(g)+μ₂(g))/2 which is the average of the mean log₁₀ expression value in a first class and a second class; x_{g} is the log₁₀ gene expression value in the sample to be tested; and wherein a positive V value indicates a vote for the first class, and a negative V value indicates a vote for the second class. In a particular embodiment the treatment outcome is survival after treatment. In another embodiment the informative genes are one or more genes described in FIGS. 1, 2A and 2B.

The invention also relates to an oligonucleotide microarray immobilized on a solid support chip, including a plurality of oligonucleotide probes specific for one or more informative genes selected from the group consisting of the genes in FIGS. 1, 2A, 2B, 3A, 3B, 4A and 4B.

The invention further relates to a method of assessing treatment efficacy in an individual having a lymphoma comprising determining the expression level of one or more informative genes at multiple time points during treatment. In one embodiment, a decrease in expression of the one or more informative genes shown to be expressed, or expressed at increased levels (as compared with a control), in individuals having a lymphoma or at risk for developing a lymphoma, is indicative that treatment is effective. In one embodiment, the lymphoma is DLCL and the one or more informative genes are selected from the group consisting of the genes in FIGS. 1, 2A, 2B, 3A, 3B, 4A and 4B.

In another embodiment, an increase in expression of the one or more informative genes shown not to be expressed, or expressed at reduced levels (as compared with a control), in individuals having a lymphoma or at risk for developing a lymphoma, is indicative that treatment is effective. In one embodiment, thelymphoma is DLCL and the one or more informative genes are selected from the group consisting of the genes in FIGS. 1, 2A, 2B, 3A, 3B, 4A and 4B.

### BRIEF DESCRIPTION OF THE FIGURES

FIG. 1 shows a list of Large B-Cell Lymphoma treatment outcome gene markers whose expression is increased in low risk and decreased in high risk individuals. The genes are identified by GenBank Accession number followed by common name.

FIGS. 2A-2B show a list of Large B-Cell Lymphoma treatment outcome gene markers whose expression is decreased in low risk and increased in high risk individuals. The genes are identified by GenBank Accession number followed by common name.

FIGS. 3A-3B show a list of informative genes whose expression is increased in DLCL and decreased in FL. The genes are identified by GenBank Accession number followed by common name.

FIGS. 4A-4B show informative genes whose expression is decreased in DLCL and increased in FL. The genes are identified by GenBank Accession number followed by common name.

### DETAILED DESCRIPTION OF THE INVENTION

The present invention relates to methods for classifying a sample according to the gene expression profile of the sample. In one embodiment, the present invention is directed to classifying a sample with respect to a phenotypic effect, e.g., lymphoma class or predicted treatment outcome, including the steps of isolating a gene expression product from one or more cells in the sample, and determining a gene expression profile of at least one informative gene, such that the gene expression profile is correlated with a phenotypic effect, thereby classifying the sample with respect to phenotypic effect.

According to the methods of the invention, samples can be classified as belonging to (i. e., derived from) a particular type of lymphoma. For example, a sample can be classified as derived from a folicular lymphoma (FL) or from a diffuse large cell lymphoma (DLCL). This distinction is not readily discernable using traditional analytic methods.

Alternatively, according to methods of the invention, samples can be classified as belonging to a particular class of treatment outcome. Such a classification sorts samples according to the likelihood of, for example, successful treatment. Samples can be sorted according to their responsiveness to drugs, therapy, or even generally to survival of the individual from whom the sample was derived. That is, a sample can be classified as belonging to a high risk class (e.g., a class with poor prognosis for survival after or without treatment) or a low risk class (e.g., a class with good prognosis for survival after or without treatment). Duration of illness, severity of symptoms and eradication of disease can also be used as the basis for differentiating, i.e., classifying, samples.

As used herein, gene expression products are proteins, peptides, or nucleic acid molecules (e.g., mRNA, tRNA, rRNA, or cRNA) that result from transcription or translation of a gene. The present invention can be effectively used to analyze proteins, peptides or nucleic acid molecules that result from transcription or translation of a particular gene or genes. Levels of gene expression can be derived directly from the levels of the gene expression products, or from measuring the activity of a corresponding regulatory gene. All forms of gene expression products can be measured, including, for example, spliced variants. Similarly, gene expression can be measured by assessing the level of protein or derivative thereof translated from mRNA. The sample to be assessed can be any sample that contains a gene expression product. Suitable sources of gene expression products, i.e., samples, can include cells, lysed cells, cellular material for determining gene expression, or material containing gene expression products. Examples of such samples are blood, plasma, lymph, urine, tissue, mucus, sputum, saliva or other cell samples. Methods of obtaining such samples are known in the art. Samples can be obtained from healthy individuals or individuals exhibiting particular phenotypes such as, for example, from an individual who has been clinically diagnosed as having a lymphoma.

In the embodiment where the gene expression product is mRNA, the gene expression levels can be obtained, for example, by contacting the sample with oligonucleotide hybridization probes contained in “microarrays.” A probe will hybridize specifically, depending on hybridization and wash conditions, such conditions being known in the art, to a specific “target” molecule. As used herein, a microarray is a known distribution of probes in known or knowable locations on, for example, a solid support chip (sometimes referred to as a “gene chip”). Such microarrays and their use are also within the scope of the invention. Examples of methods of making oligonucleotide microarrays are described, for example, in WO 95/11995. Other methods for measuring specific RNA levels in a sample are known to one of skill in the art.

In the case where the gene expression product is a protein or polypeptide, determination of the level of gene expression can be made using techniques for protein detection and quantitation known in the art. For example, antibodies specific for the protein or polypeptide can be obtained using methods that are routine in the art, and the specific binding of such antibodies to protein or polypeptide gene expression products can be detected and measured.

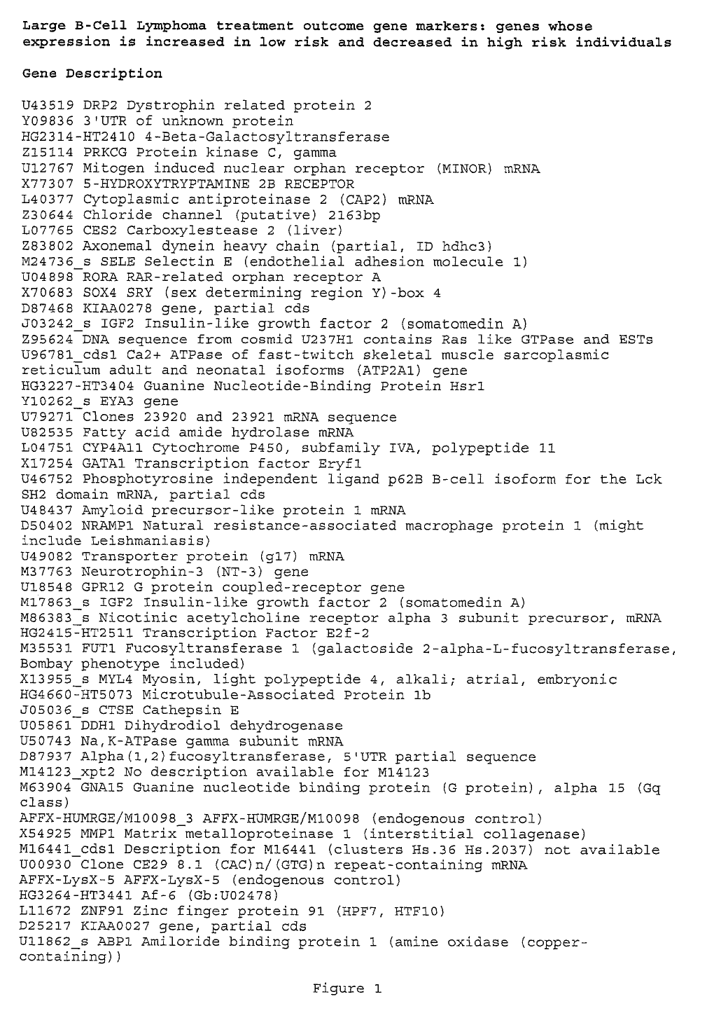

Genes that are particularly relevant for classification have been identified as a result of work described herein and are shown in FIGS. 1, 2A, 2B, 3A, 3B, 4A and 4B. The genes that are relevant for classification are referred to herein as “informative genes.” Not all informative genes for a particular class distinction must be assessed in order to classify a sample. Similarly, the set of informative genes important for one phenotypic effect may or may not be the same as the set of informative genes useful for classifying a different phenotypic effect. For example, a subset of the informative genes that demonstrate a high correlation with a class distinction can be used. This subset can be, for example, one or more genes, 5 or more genes, 10 or more genes, 25 or more genes, or 50 or more genes. Typically the accuracy of the classification will increase with the number of informative genes assessed, thus increasing the confidence level of the prediction. The particular subset of genes used to classify one phenotype might include genes that are different from the genes included in a subset of informative genes useful for classifying a different phenotype.

“Gene expression profile” as used herein is defined as the level or amount of gene expression of particular genes as assessed by methods described herein. The gene expression profile can comprise data for one or more genes and can be measured at a single time point or over a period of time. Phenotype classification (e.g., treatment outcome, lymphoma type) can be made by comparing the gene expression profile of the sample with respect to one or more informative genes with one or more gene expression profiles (e.g., in a database). Informative genes include, but are not limited to, those shown in FIGS. 1, 2A, 2B, 3A, 3B, 4A and 4B. Using the methods described herein, expression of numerous genes can be measured simultaneously. The assessment of numerous genes provides for a more accurate evaluation of the sample because there are more genes that can assist in classifying the sample.

Once the gene expression levels of the sample are obtained, the levels are compared to or evaluated against a model, and the sample is classified. The model is generated based on gene expression profiles from samples that are known. The models represent a standard against which unknown sample gene expression profiles are compared. The evaluation of a sample determines whether or not the sample is assigned to the particular phenotypic class being studied. For example, a model can be generated where expression values of the informative genes correlate with a DLCL phenotype. If the sample gene expression profile matches the model for a DLCL phenotype by the methods described herein, then the sample is classified as a DLCL sample.

The gene expression value measured or assessed is the numeric, i.e., quantitative, value obtained from an apparatus that can measure gene expression levels. Gene expression levels refer to the amount of expression of the gene expression product, as described herein. The values can be raw values from the apparatus, or values that are optionally rescaled, filtered and/or normalized. Such data is obtained, for example, from a GeneChip® probe array or Microarray (Affymetrix, Inc.)(U.S. Pat. Nos. 5,631,734, 5,874,219, 5,861,242, 5,858,659, 5,856,174, 5,843,655, 5,837,832, 5,834,758, 5,770,722, 5,770,456, 5,733,729, 5,556,752, all of which are incorporated herein by reference in their entirety), and the expression levels can be calculated with software (e.g., Affymetrix GENECHIP® software).

Nucleic acids (e.g., mRNA, cDNA, pre-mRNA) from a sample hybridize to the probes on a chip containing a DNA microarray. A sample is obtained and the nucleic acid to be analyzed (e.g., the target) is isolated, amplified and labeled with a detectable label, e.g., ³²P or a fluorescent label, prior to hybridization to the arrays. The isolated and labeled nucleic acid is allowed to contact the chip under conditions suitable for hybridization to occur. Unbound sample is washed under particular stringency conditions, and the bound nucleic acid is detected using a scanner that quantitatively detects the number of molecules hybridized to a particular probe. Since the sequence and position of each probe on the array are known, the identity and amount of the target nucleic acid applied to the probe is determined.

Quantitation of gene expression based on hybridization of labeled mRNA to DNA probes in a microarray can be performed by scanning the microarrays to measure the amount of hybridization at each position on the microarray with an Affymetrix scanner (Affymetrix, Santa Clara, Calif.). The levels of hybridization are determined by the amount of label that accumulates, via hybridization of the labeled target to the probe, at a location on a chip where a specific probe has been placed. A strong signal will occur if, for example, 90% of the probes at a particular location hybridize to labeled target, and a weaker signal will occur if, for example, only 30% of the probes present at the location hybridize to a labeled target. Strength of signal, therefore, is indicative of the amount of expression since expression of a specific target is proportional to the level of specific target-probe hybridization.

The detection of the hybridization interaction between the target and the probe can also be affected by the strength of interaction between the target and probe. The strength of interaction affects the efficiency of hybridization. For example, if the probe is perfectly complementary to the target, then strong, highly efficient hybridization interactions occur. If there are mismatches, however, between the probe and target, the hybridization interaction is weaker, and, thus, fewer interactions occur. The differences in strength of hybridization interaction can be manipulated by changing stringency conditions during hybridization and subsequent washing of the chip.

Quantification of the fluorescent signal and correlation to expression level involves examining separate stimuli specific to a particular probe and target pair. For each stimulus, a time series of mRNA levels (C={C1, C2, C3, . . . Cn}) and a corresponding time series of mRNA levels (M={M1, M2, M3, . . . Mn}) in control medium in the same experiment as the stimulus is obtained. Quantitative data are then analyzed. Cᵢ and Mᵢ are defined as relative steady-state mRNA levels, where i refers to the i^{th} time point and n to the total number of time points of the entire time course. μM and σM are defined as the mean and standard deviation of the control time course, respectively. In this way, gene expression profiles can be obtained from a single sample at different time points. The reference expression profile can be, for example, representative of expression levels at a steady state, and the sample can be taken from a time point, for example, after drug treatment.

In addition to the use of microarrays, other methods known in the art can be employed to obtain expression profiles. For example, antibodies used in immunoassays can detect peptide or protein gene expression products. Since immunoassays can be quantitative, detection of peptide or protein gene expression products will correlate with the level of gene expression.

The use of microarrays and immunoassays encompass only two classes of methods that can be used to obtain gene expression values. Other methods for obtaining gene expression values known in the art or developed in the future can be used with the present invention. Once the gene expression values are prepared, the sample can be classified.

The correlation between gene expression and class distinction can be determined using a variety of methods. Methods of defining classes and classifying samples are described, for example, in U.S. patent application Ser. No. 09/544,627, filed Apr. 6, 2000 by Golub et al., the teachings of which are incorporated herein by reference in their entirety. The information provided by the present invention, alone or in conjunction with other test results, aids in sample classification.

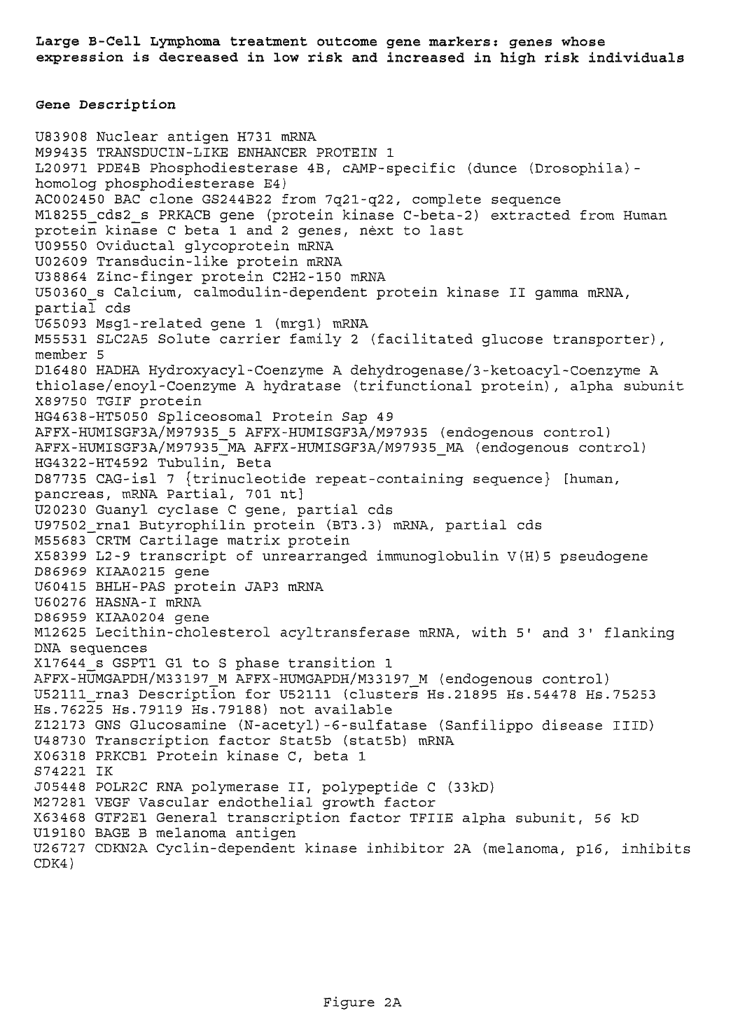

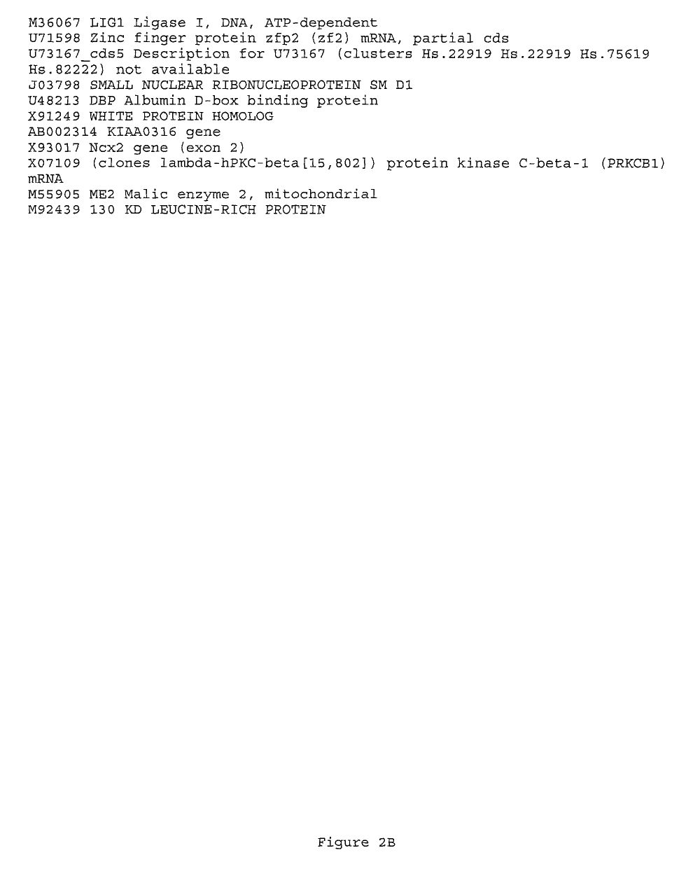

In one embodiment, the sample is classified using a weighted voting scheme. The weighted voting scheme advantageously allows for the classification of a sample on the basis of multiple gene expression values. In a preferred embodiment the sample is a lymphoma patient sample, e.g., a DLCL or FL patient sample. In a preferred embodiment the sample is classified as belonging to a particular treatment outcome class. In another embodiment the gene is selected from a group of informative genes, including, but not limited to, the genes listed in FIGS. 1, 2A and 2B.

One aspect of the present invention is directed to a method of assigning a sample to a known or putative class, e.g., a DLCL or FL treatment outcome class, comprising determining a weighted vote of one or more informative genes (e.g., greater than 10, 20, 30, 40 or 50 genes) for one of the classes in accordance with a model built with a weighted voting scheme, such that the magnitude of each vote depends on the expression level of the gene in the sample and on the degree of correlation of the gene's expression with class distinction; and summing the votes to determine the winning class. The weighted voting scheme is:

V_{g}=a_{g}(x_{g}−b_{g})

wherein V_{g} is the weighted vote of the gene, g; a_{g} is the correlation between gene expression values and class distinction, P_{(gc)}, as defined herein; b_{g}=(μ₁(g)+μ₂(g))/2, which is the average of the mean log₁₀ expression value in a first class, μ₁(g), and a second class, μ₂(g); x_{g} is the log₁₀ gene expression value in the sample to be tested; and wherein a positive V value indicates a vote for the first class, and a negative V value indicates a negative vote for the class. A prediction strength can also be determined, such that the sample is assigned to the winning class if the prediction strength is greater than a particular threshold, e.g., 0.3. The prediction strength is determined by:

(V_{win}−Vₗₒₛₑ)/(V_{win}+Vₗₒₛₑ)

wherein V_{win} and Vₗₒₛₑ are the vote totals for the winning and losing classes, respectively.

As a consequence of the identification of informative genes for the prediction of treatment outcome, the present invention provides methods for determining a treatment plan for an individual. That is, a determination of the lymphoma class or treatment outcome class to which the sample belongs may dictate that a treatment regimen be implemented. For example, once a health care provider knows to which treatment outcome class the sample belongs, and therefore, the individual from which it was obtained belongs, the health care provider can determine an adequate treatment plan for the individual. For example, in the treatment of a patient whose gene expression profile as determined by the present invention correlates with a poor prognosis, a health care provider could utilize a more aggressive treatment for the patient, or provide the patient with a realistic assessment of his or her prognosis.

The present invention also provides methods for monitoring the effect of a treatment regimen in an individual by monitoring the gene expression profile for one or more informative genes. For example, a baseline gene expression profile for the individual can be determined, and repeated gene expression profiles can be determined at time points during treatment. A shift in gene expression profile from a profile correlated with poor treatment outcome to profile correlated with improved treatment outcome is evidence of an effective therapeutic regimen, while a repeated profile correlated with poor treatment outcome is evidence of an ineffective therapeutic regimen.

The present invention also provides information regarding the genes that are important in DLCL or FL treatment response, thereby providing additional targets for diagnosis and therapy. It is clear that the present invention can be used to generate databases comprising informative genes that will have many applications in medicine, research and industry.

The invention will be further described with reference to the following non-limiting examples. The teachings of all the patents, patent applications and all other publications and websites cited herein are incorporated by reference in their entirety.

### EXEMPLIFICATION

Treatment Outcome Prediction

A gene expression-based predictor of Diffuse Large Cell Lymphoma (DLCL) patient response to treatment was built by analyzing patient samples. RNA obtained from patients was analyzed on Affymetrix (Santa Clara, Calif.) oligonucleotide arrays containing probes for 6817 genes as previously described (Tamayo et al., 1999. Proc. Natl. Acad. Sci. USA. 96:2907-2912). In addition to the weighted voting method described, a “k-Nearest Neighbors” (k-NN) algorithm was applied. The k-NN algorithm makes no assumptions about the data and “memorizes” the training set. To predict a new sample it computes the distance of the new sample to each sample in the memorized training set. Then each of the k closest samples will have an associated class. The algorithm sets the class of the new data point to the majority class appearing in the k closest training set samples.

In the molecular classification problems, one typically considers a large set of features and therefore performs a feature selection process by which the k-NN algorithm is fed only the features with higher correlation with the target class. This feature selection is done by sorting the features according to the same signal-to-noise statistic used in the weighted voting algorithm. Other variations of the algorithm, which include different ways to weight the samples in the training set, were also used. The two choices used were 1) weighting the neighbors according to Euclidean distance or 2) the rank (k) from the new sample.

As a result of these analyses a set of informative genes was identified as shown in FIGS. 1, 2A and 2B. These genes show a significant correlation with treatment outcome (e.g., patient survival). Utilizing these genes, patient survival can be predicted with high accuracy (p<0.004), even among patients within a single clinical risk group whose prognosis is otherwise indeterminate.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of classifying a lymphoma sample according to predicted treatment outcome comprising the steps of:
a) isolating a gene expression product from at least one informative gene from one or more cells in said sample; and
b) determining a gene expression profile of at least one informative gene,
wherein the gene expression profile is correlated with a treatment outcome, thereby classifying the sample with respect to treatment outcome.

2. A method according to claim 1, wherein the lymphoma sample is a diffuse large cell lymphoma sample.

3. A method according to claim 1, wherein the gene expression product is mRNA.

4. A method according to claim 3, wherein the gene expression profile is determined using hybridization probes specific to at least one informative gene.

5. A method according to claim 3, wherein the gene expression profile is determined using oligonucleotide microarrays.

6. A method according to claim 1, wherein the gene expression product is a peptide.

7. A method according to claim 6, wherein the gene expression profile is determined using antibodies.

8. A method according to claim 1, wherein the predicted treatment outcome is survival after treatment.

9. A method according to claim 1, wherein the informative gene is selected from the group consisting of the genes in FIG. 1.

10. A method according to claim 1, wherein the informative gene is selected from the group consisting of the genes in FIGS. 2A and 2B.

11. A method of assigning a diffuse large cell lymphoma sample to a treatment outcome class, comprising the steps of:
a) determining a weighted vote for one of the classes of one or more informative genes in said sample in accordance with a model built with a weighted voting scheme, wherein the magnitude of each vote depends on the expression level of the gene in said sample and on the degree of correlation of the gene's expression with class distinction; and
b) summing the votes to determine the winning class,
wherein the winning class is the treatment outcome class to which the diffuse large cell lymphoma sample is assigned.

12. The method of claim 11, wherein the weighted voting scheme is:
V_{g}=a_{g}(x_{g}−b_{g}),
wherein V_{g} is the weighted vote of the gene, g; a_{g} is the correlation between gene expression values and class distinction; b_{g}=(μ₁(g)+μ₂(g))/2, which is the average of the mean log₁₀ expression value in a first class and a second class; x_{g} is the log₁₀ gene expression value in the sample to be tested; and wherein a positive V value indicates a vote for the first class, and a negative V value indicates a vote for the second class.

13. The method according to claim 11, wherein the informative genes are selected from the group consisting of the genes in FIG. 1.

14. The method according to claim 11, wherein the informative genes are selected from the group consisting of the genes in FIGS. 2A and 2B.

15. A method of classifying a sample according to lymphoma type comprising the steps of:
a) isolating a gene expression product from at least one informative gene from one or more cells in said sample; and
b) determining a gene expression profile of at least one informative gene,
wherein the gene expression profile is correlated with a lymphoma type, thereby classifying the sample with respect to lymphoma type.

16. A method according to claim 15, wherein the lymphoma type is diffuse large cell lymphoma.

17. A method according to claim 15, wherein the lymphoma type is follicular lymphoma.

18. A method according to claim 15, wherein the gene expression product is mRNA.

19. A method according to claim 18, wherein the gene expression profile is determined using hybridization probes specific to one ore more informative genes.

20. A method according to claim 18, wherein the gene expression profile is determined using oligonucleotide microarrays.

21. A method according to claim 15, wherein the gene expression product is a peptide.

22. A method according to claim 21, wherein the gene expression profile is determined using antibodies.

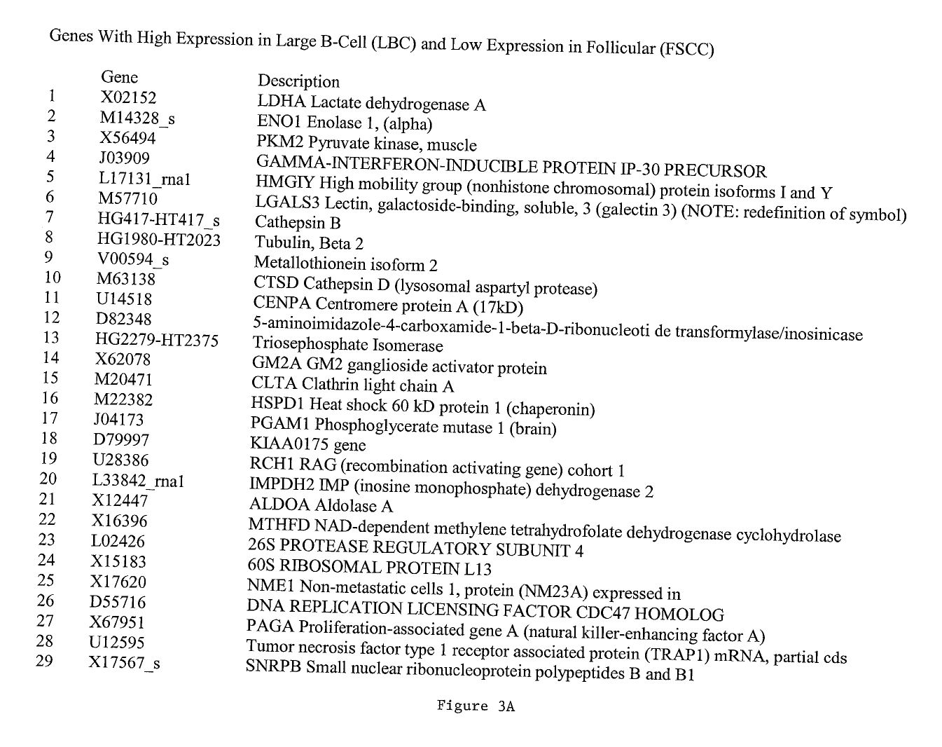

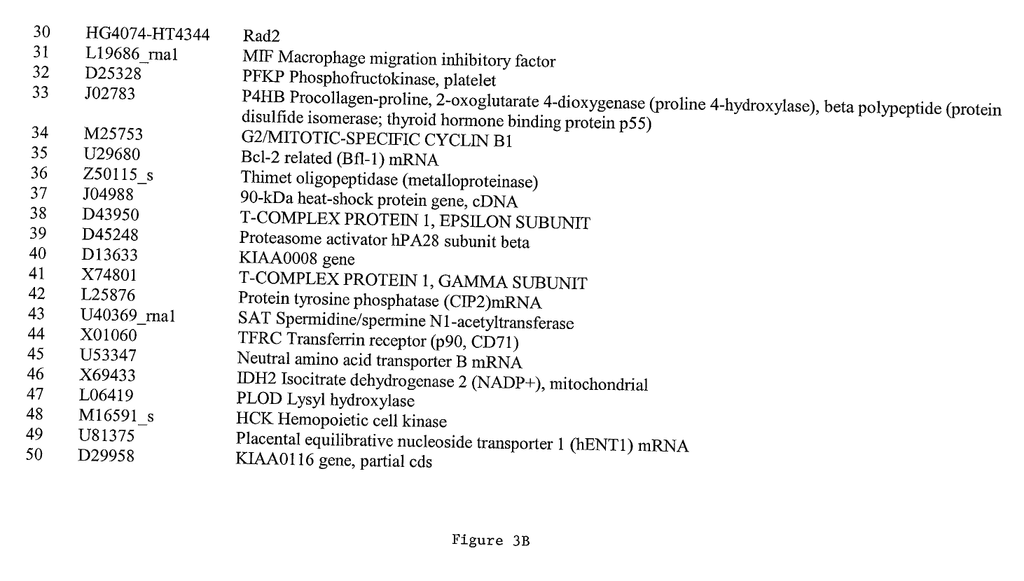

23. A method according to claim 15, wherein the informative gene is selected from the group consisting of the genes in FIGS. 3A and 3B.

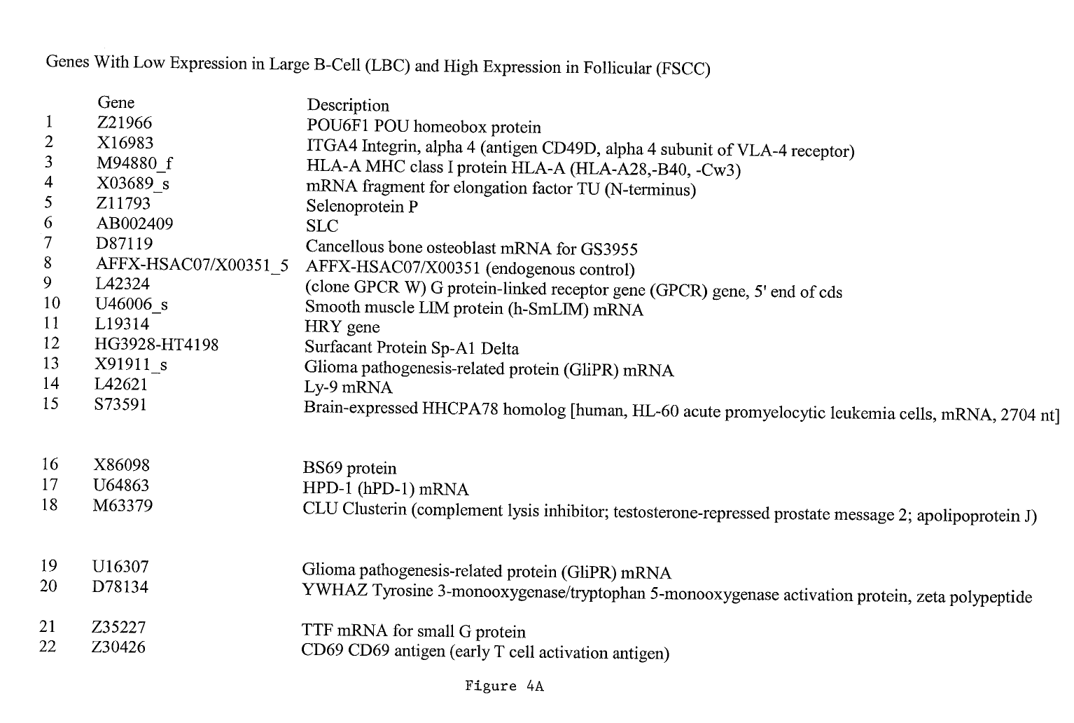

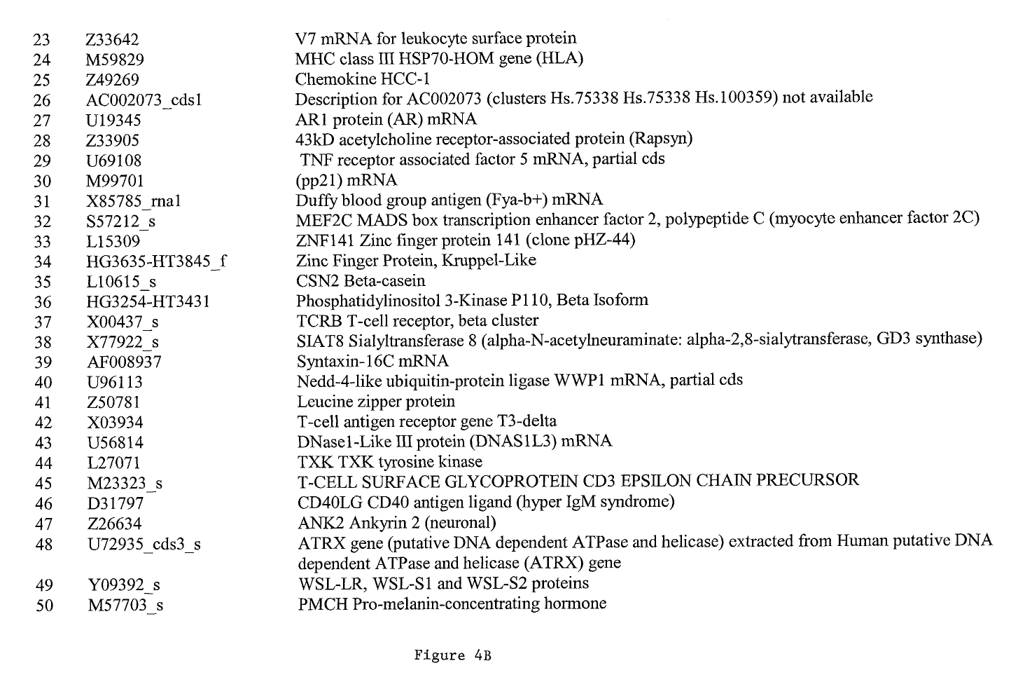

24. A method according to claim 15, wherein the informative gene is selected from the group consisting of the genes in FIGS. 4A and 4B.

25. A method of assigning a sample to a lymphoma class, comprising the steps of:
a) determining a weighted vote for one of the classes of one or more informative genes in said sample in accordance with a model built with a weighted voting scheme, wherein the magnitude of each vote depends on the expression level of the gene in said sample and on the degree of correlation of the gene's expression with class distinction; and
b) summing the votes to determine the winning class,
wherein the winning class is the lymphoma class to which the lymphoma sample is assigned.

26. The method of claim 25, wherein the weighted voting scheme is:
V_{g}=a_{g}(x_{g}−b_{g}),
wherein V_{g} is the weighted vote of the gene, g; a_{g} is the correlation between gene expression values and class distinction; b_{g}=(μ₁(g)+μ₂(g))/2, which is the average of the mean log₁₀ expression value in a first class and a second class; x_{g} is the log₁₀ gene expression value in the sample to be tested; and wherein a positive V value indicates a vote for the first class, and a negative V value indicates a vote for the second class.

27. The method according to claim 25, wherein the informative genes are selected from the group consisting of the genes in FIGS. 3A and 3B.

28. The method according to claim 25, wherein the informative genes are selected from the group consisting of the genes in FIGS. 4A and 4B.

29. An oligonucleotide microarray immobilized on a solid support chip, comprising a plurality of oligonucleotide probes specific for one or more informative genes selected from the group consisting of the genes in FIGS. 1, 2A, 2B, 3A, 3B, 4A and 4B.

30. A method of assessing treatment efficacy in an individual having a lymphoma comprising determining the expression level of one or more informative genes at multiple time points during treatment.

31. The method of claim 30, wherein a decrease in expression of the one or more informative genes shown to be expressed, or expressed at increased levels as compared with a control, in individuals having a lymphoma or at risk for developing a lymphoma, is indicative that treatment is effective.

32. The method of claim 31, wherein the lymphoma is a diffuse large cell lymphoma.

33. The method of claim 31, wherein the one or more informative genes are selected from the group consisting of the genes in FIGS. 1, 2A, 2B, 3A, 3B, 4A and 4B.

34. The method of claim 30, wherein an increase in expression of the one or more informative genes shown not to be expressed, or expressed at reduced levels as compared with a control, in individuals having a lymphoma or at risk for developing a lymphoma, is indicative that treatment is effective.

35. The method of claim 34, wherein the lymphoma is a diffuse large cell lymphoma.

36. The method of claim 34, wherein the one or more informative genes are selected from the group consisting of the genes in FIGS. 1, 2A, 2B, 3A, 3B, 4A and 4B.
